(19) United States
(12) Patent Application Publication
(10) Pub. No.: US 2007/0093457 A1
(43) Pub. Date: Apr. 26, 2007

(54) Compositions for treatment of cancer and inflammation with curcumin and at least one nsaid

(76) Inventors: Nadir Arber, Tel Aviv (IL); Shahar Lev-Ari, Tel Aviv (IL); Dov Lichtenberg, Tel Aviv (IL)

Correspondence Address: NATH & ASSOCIATES, 112 South West Street, Alexandria, VA, 22314, US

(21) Appl. No.: 10/589,000

(22) PCT Filed: Feb. 10, 2005
(86) PCT No.: PCT/IL05/00173

§ 371 Date: Nov. 17, 2006

Related U.S. Application Data
(60) Provisional application No. 60/543,389, filed on Feb. 11, 2004.

(51) Int. Cl. A61K 31/60; A61K 31/415; A61K 31/405; A61K 31/122; A61K 31/12

(52) U.S. Cl. 514/165; 514/406; 514/420; 514/569; 514/570; 514/688

(57) ABSTRACT

A composition is provided which comprises an effective amount of curcumin and an effective amount of at least one NSAID for the treatment of cancer and inflammatory diseases and disorders. Also provided are methods of treatment of such diseases and disorders.

### FIELD OF THE INVENTION

This invention relates in general to the field of cancer and anti-inflammatory treatment.

### BACKGROUND OF THE INVENTION

Cyclooxygenase, herein designated as “COX” or prostaglandin H synthase is the enzyme that catalyzes rate-limiting steps in the biosynthesis of prostaglandins (PGs). In contrast to COX-1, the constitutive form that plays an important role in cell homeostasis, COX-2, also known as the “bad COX”, is the inducible form and is mainly involved in the onset of inflammation and mitogenic responses (Dubois et al., FASEB J. 12, 1063-1073, 1998; Williams et al., Oncogene, 18, 7908-7916, 1999). Since the identification and cloning of the COX-2 gene, accumulating evidence supports the critical role of COX-2 in carcinogenesis. Upregulation of COX-2 expression and PG production are commonly found in many cancer cells such as colorectal cancer. A number of COX-2 inhibitors such as the non-steroidal and anti-inflammatory drugs (NSAIDs) are able to block the COX enzymes and reduce PGs throughout the body and thus induce growth inhibition in cancer cells. They also inhibit angiogenesis and can reduce ongoing inflammation, pain and fever.

Several mechanisms have been proposed to explain why COX-2 expression in neoplastic tissue enhances tumor growth. There is evidence suggesting that COX-2-produced PGE₂ causes amplification of tumor cell proliferation, inhibition of tumor cell apoptosis, enhancement of stromal cell angiogenesis and decreased immune surveillance of tumor cells.

Celecoxib, also known as Celebrex, is the foremost branded non-steroidal and anti-inflammatory drug and the leading COX-2 specific inhibitor. Celecoxib has been shown to provide relief of the pain and inflammation of osteoarthritis, adult rheumatoid arthritis, acute pain, and primary dysmenorrheal in adults. In addition celecoxib has been shown to reduce the number of adenomatous colorectal polyps in familial adenomatous polyposis.

Celecoxib has been used in various combinations with other substances. One such example is disclosed in U.S. Pat. No. 6,573,290 to Love in which a method for treating cancers comprising the administration of a combination of celecoxib and alpha-difluoromethylomithine (DFMO) to a patient in need thereof is disclosed.

Aggarwal et al., (Apoptosis, 8, 649-654, 2003) has used SC236, a structural analogue of celecoxib, in combination with curcumin and showed that such combination may auginent their selectivity for COX-2 overexpressing colorectal cancer cells and potentially could be more effective than SC236 or celecoxib when used alone in colon cancer chemoprevention. In this study Aggarwal et al utilized very high, and potentially toxic, dosages of the NSAIDs.

Curcumin has been used in combination with other drugs as well and showed COX-2 inhibition activity. US Patent Application No. 2003/0108628 discloses compositions comprising curcuminoid species such as curcumin along with diterpene lactones that in combination exhibit a synergistic effect on specific inhibition of inducible COX-2 activity and have minimal effect on COX-1 activity.

Unlike celecoxib which is thought to cause adverse effects that might militate against long-term administration, curcumin is a natural product of low toxicity. Curcumin inhibits COX-2 at the transcriptional level in cells in vitro. Although it displays low bioavailability, concentrations inhibitory to COX-2 are attainable in the colonic mucosa of rats after dietary administration. However, the doses needed to achieve such tissue concentrations far exceed those normally consumed as turmeric in the diet. Curcumin, known to inhibit various signaling pathways, is an important player in the induction of expression of the gene for COX-2 by inflammatory and tumor-promoting stimuli. It is thus likely to inhibit transcription of the gene at more than one level.

### SUMMARY OF THE INVENTION

The present invention is based on the surprising observations that a combination of curcumin and an NSAID, wherein the concentration of said NSAID drug is lower than the normally effective concentration to treat a certain condition, showed at least one of the desired therapeutic effects, additive or synergistic, in vivo or in vitro. This observation paves the way to the use of smaller dosages of each compound, and in particular smaller amounts of the high and toxic NSAID such as celecoxib, thereby improving the safety profile of this regimen.

Thus, by one aspect of the present invention, there is provided a method for reducing a dosage size of an at least one NSAID in the treatment of a patient in need of an NSAID therapy, comprising simultaneous or step-wise administering of curcumin and said at least one NSAID, the curcumin being in an amount sufficient to reduce the NSAID concentration needed while maintaining the same therapeutic effect as compared to administering the NSAID alone.

By the term “reducing a dosage size” is meant reducing the amount or concentration of said at least one NSAID by using curcumin by at least about 5%, 10%, 20%, 30%, 40%, 50%, 60%, 70%, 80%, 90% or 95% as compared to the amount or concentration of said at least one NSAID in a single dosage, if administered alone for the treatment of the same disease or disorder. The preferred reduced dosage is one which emphasizes therapeutic benefits but minimizes short or long term adverse effects such as gastroduodenal ulcers, strictures, esophagitis, gastritis, colitis, small and large bowel erosions, acute and/or chronic renal failure, fluid and electrolyte imbalances, hyperflalemia, hematuria, nephrotic syndrome with interstitial nephritis, papillary necrosis, exacerbation of hypertension, exacerbation of congestive heart failure, arrhythmia, elevated transaminases, choleostasis, hepatic failure, headache, tinnitus, vertigo, tremor, depression, somnalence, altered mental status, aseptic meningitis, thrombocytopenia, hemolytic anemia, agranulocytosis, leukopenia, eosinophilia, aplastic anemia, exacerbation of asthma, cough, respiratory depression, laryngeal and pharyngeal edema, skin rash, photosensitivity, Stevens Johnson syndrome, pemphigoid reaction, erythema multiform, urticaria, angioedema, joint erosions or decreased repair of cartilage damage.

By “simultaneous” it is meant as a single therapeutic dosage form, e.g., as a single capsule, tablet or an injection which comprises both curcumin and at least one NSAID. The term “step-wise” refers to two separate therapeutic dosages such as in separate capsules, tablets or injections. The two separate dosages, as will be described hereinbelow, may be administered for example one immediately after the other, one before and one after meal, one in the morning and one in the afternoon, or in any other regimen that the practitioner prescribing the drug combination may find suitable based on the condition of the subject and any other parameters.

The term “curcumin” refers broadly to any analogue, derivative, or salt thereof, such as demethoxycurcumin and bisdemethoxycurcumin, which may be synthetic or natural. Also included under this term is any botanical member of the curcuminoid family such as gingerol. By “NSAID” is meant any member of the class of compounds, or derivatives, analogues, salts or prodrug thereof, which are characterized as being non-steroidal and anti-inflammatory drugs. These may be, without limiting the invention thereto, ketorolac, nabumetone, salsalate, diclofenac, indomethacin, nabumetone, phenylbutazone, oxyphenbutazone, dipyrone, ramifenazone, tenoxicam, valdecoxib, parecoxib, etoricoxib, celecoxib, sulindac, sulindac sulfide, exisulind, ibuprofen, naproxen, naproxen sodium, rofecoxib, nimesulide, aspirin, tolmetin, fenoprofen, flurbiprofen, loxoprofen, vedaprofen, meclofenamic acid, meclofenamate sodium, tolfenamic acid, acetaminophen, flunixin, piroxicaam, oxaprozin, meloxicam, ketoprofen, etodolac, diflunisal and the like or any derivative, analogue, salt or prodrug thereof.

In a preferred embodiment the NSAID is celecoxib, nimesulide, sulindac or sulindac sulfide or a derivative, analogue, salt or prodrug thereof. In another embodiment the NSAID is a drug other than celecoxib, or a drug other than sulindac, or a drug other than sulindac sulfide or a derivative, analogue, salt or prodrug thereof.

The expression “an NSAID therapy” refers to therapy administered to a subject suffering from a disease or disorder, which therapy makes use of one or more NSAID as means for treating said disease or disorder. Examples of diseases or disorders include without being limited thereto cancers and inflammation such as arthritis, rheumatoid arthritis, osteoarthritis, and pericarditis, and such painful conditions as back pain, sciatica, sprains, strains, dental pain, post-operative pain, period pain (dysmenorrhoea) and heavy periods (menorrhagia), pain from kidney stones, migrane, Alzheimer, for reduction of fever and other painful conditions.

Generally, the term “treatment” or any lingual variation thereof refers to the application of the remedy to a subject in need thereof, said application resulting in one or more “therapeutic effects” such as amelioration of undesired symptoms associated with the condition, prevention of the manifestation of the symptoms before they occur, slowing down the progression of the condition, slowing down the deterioration of symptoms, enhancement of onset of remission period, slowing down the irreversible damage caused by the condition, delaying the onset of said progressive stage, lessening of severity or curing the condition, or prevention of the condition from reoccurring or a combination of two or more of the above.

By another aspect, the present invention provides the use of a combination of curcumin and at least one NSAID for the manufacture of a pharmaceutical composition.

In one embodiment, the combination of curcumin and at least one NSAID is used for the manufacture of a pharmaceutical composition in which said curcumin is in an amount sufficient to reduce the amount of said at least one NSAID needed while maintaining the same therapeutic effect as compared to administering the NSAID alone.

In another embodiment, said use is for the manufacture of anti-cancer or anti-inflammation composition.

By another aspect, the present invention provides the use of at least one NSAID for the preparation of a pharmaceutical composition for the treatment of cancer or inflammation in a subject, said composition is to be administered simultaneously with a treatment with curcumin. The composition comprising said NSAID may also be administered following a treatment with curcumin.

By yet another aspect, the present invention provides the use of curcumin for the preparation of a pharmaceutical composition for the treatment of cancer or inflammation in a subject, said composition is to be administered simultaneously with a treatment with curcumin. The composition comprising said NSAID may also be administered following a treatment with curcumin.

By yet another aspect, the present invention provides a pharmaceutical composition comprising curcumin and at least one NSAID and a pharmaceutically acceptable carrier, excipient or diluent.

In one embodiment said composition comprises curcumin is in an amount sufficient to reduce the amount of said at least one NSAID needed while maintaining the same therapeutic effect as compared to administering the NSAID alone.

In another embodiment of the present invention, the composition is used for the treatment of any such disease or disorder treatable with at least one NSAID is administered alone. Preferably, the disease or disorder is cancer or inflammation.

In still another aspect of the present invention, there is provided a pharmaceutical composition for the treatment of cancer or inflammation, comprising one of curcumin and at least one NSAID to be administered simoultanously with or following treatment with a second of curcumin and at least one NSAID. For example, said treatment may involve administering to a subject in need thereof an amount of at least one NSAID, and thereafter administering an amount of curcumin; or said treatment may involve administering to a subject in need thereof an amount of curcumin and thereafter administering an amount of at least one NSAID. Such composition may comprise an effective amount of each of said curcumin or at least one NSAID together with a pharmaceutically acceptable carrier, excipient or diluent.

Also provided is a combination of two pharmaceutical compositions including a first composition comprising an effective amount of at least one NSAID, and a second composition comprising an effective amount of curcumin, the combination is intended for administering to a subject for treatment of cancer or inflammation, in which treatment said second composition is administered after administering said first composition. The first composition may also be administered after said second composition. The combination, as will be disclosed hereinbelow, may be packaged to include both compositions.

The term “cancer” refers to all types of cancer or neoplasm or malignant tumors found in mammals, including leukemia, carcinomas and sarcomas. Examples of cancers are cancer of the brain, breast, cervix, colon, head and neck, kidney, lung, non-small cell lung, melanoma, mesothelioma, ovary, sarcoma, stomach, uterus and Medulloblastoma. By “leukemia” is meant broadly to progressive, malignant diseases of the blood-forming organs and is generally characterized by a distorted proliferation and development of leukocytes and their precursors in the blood and bone marrow.

Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In a most preferred embodiment, the composition of the present invention is used for the treatment of colorectal or colon cancer.

In another embodiment, the compositions or formulations comprising the combination of the present invention are useful for inhibiting the growth of a cell derived from a cancer or neoplasm such as those disclosed hereinabove. Preferably, these are useful for inhibiting the growth of cells derived from cancer such as colorectal, pancreas, and stomach.

The term “inflammation” refers in the context of the present invention to any disease wherein a response to insult, followed by invasion of leukocytes (notably monocytes and lymphocytes) to the insult site, is trailed by massive secretion of cytokines and various other cell mediators. Examples of inflammation related diseases or disorders are arthritis, including rheumatoid arthritis (RA), spondyloathopathies, gouty arthritis, osteoarthritis, systemic lupus erythematosus and juvenile arthritis. The term also encompasses immuno-inflammatory diseases including autoimmune diseases, proliferative skin diseases and any other disorders which result in the destruction of healthy tissue by an inflammatory process, dysregulation of the immune system and unwanted proliferation of cells. Non-limiting examples of immuno-inflammatory disorders are acne vulgaris, acute respiratory distress syndrome, Addison's disease, allergic rhinitis, allergic intraocular inflammatory diseases, ANCA-associated small-vessel vasculitis, athrosclerosis, atopic dermatitis, autoimmune hemolytic anemia, autoimmune hepatitis, Behcet's disease, Bell's palsy, cerebral ischemia, Cogan's syndrome, dermatomyositis and psoriatic arthritis.

The compositions may also be used to, menstrual cramps, tendonitis, bursitis, Crohn's disease, Alzheimer and ulcerative colitis.

In another most preferred embodiment, the composition is used for the treatment of arthritis.

As used herein the term “pharmaceutical composition” refers to a preparation of one or more of the components described herein, or physiologically acceptable salts or prodrugs thereof, with other chemical components such as physiologically suitable carriers and excipients. The purpose of a pharmaceutical composition is to facilitate administration of a compound to an organism. The term “prodrug” refers a precursor compound that can hydrolyze, oxidize, or otherwise react under biological conditions (in vitro or in vivo) to provide the active compound, namely said at least one NSAID. Examples of prodrugs include, but are not limited to, metabolites of said NSAID that include biohydrolyzable moieties such as biohydrolyzable ainides, biohydrolyzable esters, biohydrolyzable carbarnates, biohydrolyzable carbonates, biohydrolyzable ureides, and biohydrolyzable phosphate analogues.

The term “excipient” refers to an inert or inactive substance added to a pharmaceutical composition to further facilitate administration of a compound. Non-limiting examples of excipients include calcium carbonate, calcium phosphate, various sugars and types of starch, cellulose derivatives, gelatin, vegetable oils and polyethylene glycols.

The pharmaceutical compositions of the present invention may also include one or more additional active ingredients, such as, but not limited to, antibiotics, conventional anti-cancer or anti-inflammatory agents or any other active ingredient that may be suitable for combination therapy. Examples of anti-cancer compounds that may be included in the compositions of the present invention are: antisense sequences, Tretinoin (Vesanoid®); Interferon (IFN)-alpha; Antineoplastics; Pegaspargase (Oncaspar®); L-asparaginase; Edatrexate; 10-ethyl-10-deaza-aminopterin; 5-fluorouracil; Levamisole; Interleukin-2 (Proleukin®); Axcan; Methyl-chloroethyl-cyclohexyl-nitrosourea; Fluorodeoxyuridine; Vincristine; Porfimer Sodium (Photofrin®); Irinotecan (Camptosar®); Topotecan (Hycamtin®); Loperamide (Imodium®); Docetaxel (Taxotere®); Rituximab; Etoposide; Faulding; Vinorelbine Tartrate (Navelbine®); Paitaxel (Taxol®); Docetaxel (Taxotere®); Irinotecan; Gemcitabine; Gemcitabine (Gemzar®); Amifostine (Ethyol®); 2-ethylhexyl-p-methoxy-cinnamate; Prednisone (Deltasone®); Octyl-N-dimethyl-paminobenzoate; Benzophenone-3; Flutamide (Eulexin®); Finasteride (Proscar®); Terazosin (Hytrin®); Doxazosin (Cardura®); Goserelin Acetate (Zoladex®); Liarozole; Nilutamide (Nilandron®); Mitoxantrone (Novantrone®); Gemcitabine (Geinzar®); Porfimer Sodium; Dacarbazine; Etoposide; Faulding; Procarbazine HCl; Rituximab; Trastuzumab (Herceptin®); and Temozolomide (Temodal®).

The pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

The term “administration” or any lingual variation thereof as used herein is meant any way of administration. The curcumin and at least one NSAID may be administered in one therapeutic dosage form or in two separate therapeutic dosages such as in separate capsules, tablets or injections. In the case of the two separate therapeutic dosages, the administration may be such that the periods between the administrations vary or are determined by the practitioner. It is however preferred that the second drug is administered within the therapeutic response time of the first drug. The curcumin and at least one NSAID which may be administered either at the same time, or separately, or sequentially, according to the invention, do not represent a mere aggregate of known agents, but a new combination with the valuable property that the effectiveness of the treatment is achieved at a much lower dosage of said at least one NSAID.

The pharmaceutical compositions of the present invention may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local.

Various delivery systems are known, e.g., encapsulation in liposomes, microparticles, microcapsules or capsules, that may be used to administer the compositions of the invention. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically to the cars, nose, eyes, or skin. The preferred mode of administration is left to the discretion of the practitioner, and will depend in part upon the site of the medical condition (such as the site of cancer) and the severity of thereof.

For example, for injection the composition of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the composition can be formulated readily by combining the active components with any pharmaceutically acceptable carriers known in the art. Such “carriers” may facilitate the manufacture of such as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Dyestuffs or pigments may be added to the tablets or dragee coatings for identification or to characterize different combinations of active NSAID doses. In addition, stabilizers may be added.

Pharmaceutical compositions for parenteral administration include aqueous solutions of the active preparation in a water-soluble form. Additionally, suspensions of the active preparation may be prepared as oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl, cellulose, sorbitol or dextran. Optionally, the suspension may also contain suitable stabilizers or agents, which increase the solubility of the compounds, to allow for the preparation of highly concentrated solutions.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

Each of the tablets or capsules of the present invention preferably contains between about 1-8 gram of curcumin and 50-400 mg of celecoxib, or equivalent dose of NSAIDs. As used herein the term “about” refers to ±20%.

The term “treatment of cancer” or any lingual variation thereof refers to at least one of the following: decrease in the rate of growth of the cancer (i.e. the cancer still grows but at a lower rate); cease of cancer growth, i.e. stasis of the cancer tumor occurs, and, in preferred cases, the cancer tumor diminishes or is reduced in size. The term also concerns reduction in the number of metastasis, reduction in the number of new metastasis formed, slowing of the progression of the cancer from one stage to the other and decrease in angiogenesis induced by the cancer. In most preferred cases, the cancer tumor is totally diminished. This term also concerns prevention for prophylactic situations or for those patients susceptible to contracting cancer; the administration of said compounds will reduce the likelihood of the individuals contracting the disease. In preferred situations, the individual to whom the compound is administered does not contract disease.

Thus, in another aspect of the present invention, there is provided a prophylactic formulation comprising curcumin and at least one NSAIDs and a pharmaceutically acceptable carrier, excipient or a diluent. The formulation may be used in the prevention of cancer or in the reduction of the likelihood of contracting cancer in a subject susceptible to contracting said disease.

The term “reduction of inflammation” or “treatment of inflammation” or any lingual variation thereof refers to at least one of the following: decrease in the number of infiltrating leukocytes, and in particular monocytes and lymphocytes to the site of injury; decrease in the amount of cytokines and mediators secreted by the infiltrating cells, decrease in at least one clinical manifestation of inflammation such as swelling, redness, pain, restriction of movement, fever, warmth, etc.

In another aspect of the present invention there is provided a method for treating cancer in a patient in need thereof, said method comprising administering to said patient an effective amount of a pharmaceutical composition comprising curcumin and at least one NSAID and a pharmaceutically acceptable carrier, excipient or diluent.

In another aspect, there is provided a method for treating an inflammatory disease or disorder in a patient in need thereof, said method comprising administering to said patient an effective amount of a pharmaceutical composition comprising curcumin and at least one NSAID and a pharmaceutically acceptable carrier, excipient or diluent.

In one preferred embodiment the NSAID is celecoxib, nimesulide, sulindac or sulindac sulfide or derivatives, analogues, salts or prodrugs thereof. In another embodiment the NSAID is a drug other than celecoxib, or a drug other than sulindac, or a drug other than sulindac sulfide or derivatives, analogues, salts or prodrugs thereof.

The term “effective amount” as used herein refers to the amount of the composition of the invention present in the composition containing thereof, which is required to treat or prevent the disease or disorder in a clinically relevant manner. A sufficient amount of the active combination used to practice the invention for therapeutic treatment of conditions caused by or contributing to said disease or disorder varies depending on the manner of administration, the age of the patient, body weight, and the general health of the patient. The practitioners will decide the appropriate amount and dosage of the regimen. Preferably, the effective amount would be one that is lower than the effective amount of each of the components when administered individually.

In yet another aspect of the present invention there is provided a method for the inhibition of cancer cell growth, that is both more efficient and significantly safer then each drug alone. The method comprises contacting cancer cells with an effective amount of a formulation comprising curcumin and at least one NSAID. In one case, the cells are contacted with a single formulation comprising both curcumin and at least one NSAID, and in another case, the cells are contacted with a formulation containing one of the components, e.g. curcumin, followed by contacting with the other, e.g., at least one NSAID, or vice versa.

The term “inhibition of cancer cells growth” as used in the present application refers to at least one of the following: decrease in the number of cells (due to cell death which may be necrotic, apoptotic or a combination thereof) as compared to the control, decrease in tumor size, decrease in rate of tumor growth, inhibition of proliferation, stasis of tumor size, decrease in the number of metastasis, decrease in the number of additional metastasis, decrease in the invasiveness of the cancer, decrease in the rate of progression of the tumor from one stage to the next as well as decrease in the angiogenesis induced by the cancer.

In yet another aspect, there is provided a method for the treatment of cancer or inflammation comprising administering to an individual in need of an effective amount of curcumin and an effective amount of at least one NSAID, the amounts being such so as to yield a synergistic combined effect of the two.

The term synergistically “effective amount” in the context of the present invention, refers to an amount which is both therapeutically active (with the other component) and shows synergistic effect as will be explained bellow. A therapeutically effective amount is determined by such considerations as may be known in the art. The amount must be effective to achieve the desired therapeutic effect as described above (i.e. treatment of cancer, reduction in cancer cell growth, reduction in inflammation), inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the distribution profile within the body, a variety of pharmacological parameters such as half life in the body, on undesired side effects, if any, on factors such as age and gender, etc. The effect is in relation to the described under the terms “reduction of growth of cancer cells”, “treatment of cancer” or “reduction of inflammation”.

The synergistic amount of curcumin or an analogue or derivative thereof used in the method of the invention, refers to concentrations of 70%, 60%, 50%, 40%, 30%, 20%, 10% or 5% w/w as compared to the total weight of the active ingredient administered without the carrier (i.e. when “70%” is mentioned it refers to 70% of celecoxib and 30% of curcumin as well as to 70% of curcumin and 30% of celecoxib). Mid-way concentrations such as 63% or 34.2% or smaller or higher concentrations are also encompassed within the scope of the invention. Additionally, it should be clear that the ratio of curcumin to the at least one NSAID may be presented by way of molar ratio or weight ratio or by any other known unit rather then percent w/w ratio.

The concentration of the curcumin needed to achieve the therapeutic effect as compared to administering the at least one NSAID alone, may be measured by testing several varying combinations of curcumin and the at least one NSAID. Any combination containing curcumin and the at least one NSAID, for which the NSAID concentration is low, yet maintaining therapeutic efficacy, as compared to concentrations of NSAID alone, in in vitro, in vivo and clinical tests, would be considered as the combination obtained by the method above.

The present invention further concerns a method for the treatment of cancer comprising administering to an individual in need of prevention or treatment of anti-cancer therapy an effective amount of curcumin and an effective amount of celecoxib, so that the effect in the treatment of cancer is higher than the sum of effects on reduction of cancer growth cell when curcumin or celecoxib are administered separately.

The present invention also concerns a method for the reduction of inflammation comprising administering to a patient in need of as an anti-inflammatory treatment an effective amount of curcumin or an analogue or derivative thereof and an effective amount of celecoxib, so that the effect in reduction of inflammation is higher than the sum of effects on reduction of inflammation when curcumin or celecoxib are administered separately.

By another aspect, the present invention provides a kit or a commercial package comprising a dosage unit of the composition of the present invention and a pharmaceutically acceptable carrier. As used herein, a “dosage unit” is a pharmaceutical composition or formulation comprising at least one of curcumin and of said at least one NSAID and optionally one or more inactive ingredient. The dosage unit can be unitary, such as a single pill or liquid, containing both curcumin and said at least one NSAID and the inactive ingredients necessary and desired for making a dosage suitable for administration (e.g., tabletting compounds such as binders, fillers, and the like); or the dosage unit can consist of a number of different dosage forms (e.g., pills, liquids etc.) designed to be taken simultaneously or sequentially as a dosage unit.

The contents of the kit may also be in the form of a powder and the kit may thus additionally contain a suitable solvent for reconstitution. Individual components of the kit may be packaged in separate containers and, associated with such containers, may be instructions for use or a legal notice in the form prescribed by a governmental agency regulating the manufacture, use or sale of pharmaceuticals.

The kit may also comprise in separate vials or containers additional active ingredients to be administered simultaneously or sequentially with the main components. The kit may be directed to the use of a medical practitioner, e.g. a doctor or a nurse or to the use of the subject in need of the composition.

Other features and advantages of the present invention will become apparent from the following detailed description. It should be understood, however, that the detailed description and the specific examples, while indicating preferred embodiments of the invention are given by way of illustration only, since various modifications within the spirit and scope of the invention will become apparent to those skilled in the art from this detailed description. Such modifications are also intended to be within the scope of the invention.

### DETAILED DESCRIPTION OF THE INVENTION

In order to understand the invention and to see how it may be carried out in practice, some preferred embodiments will now be described, by way of non-limiting examples only, with reference to the accompanying drawings, in which:

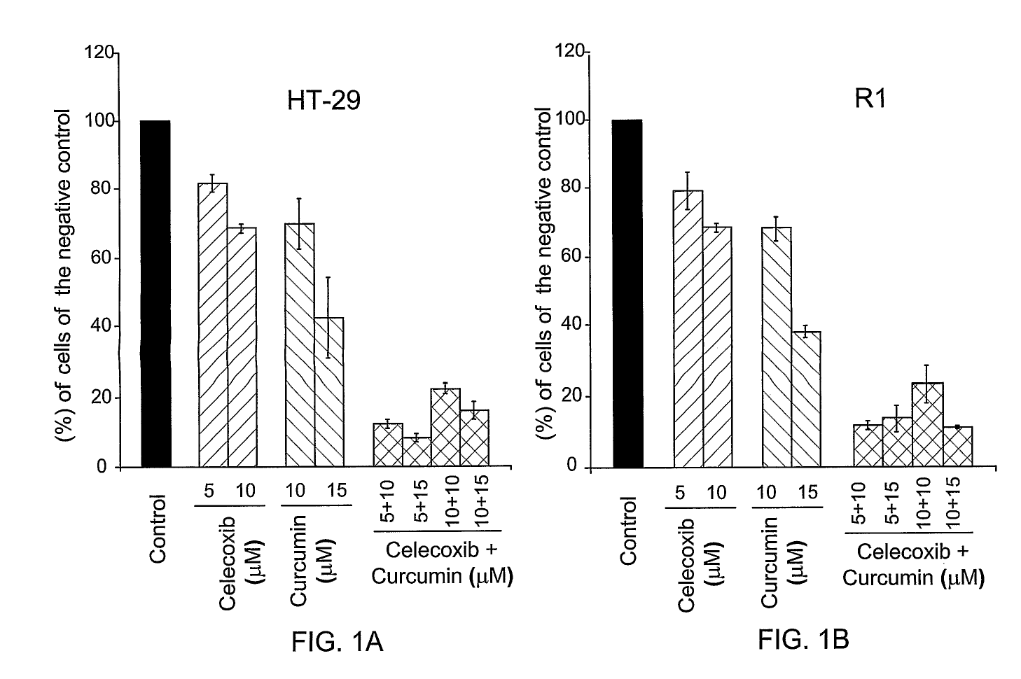

FIGS. 1A and 1B depict the effect of celecoxib and curcumin on cell growth of the human colon carcinoma cell line HT-29 (FIG. 1A) and the c-K-ras-transformed rat intestinal epithelial cell line (IEC18-ras, designated in FIG. 1B as R1).

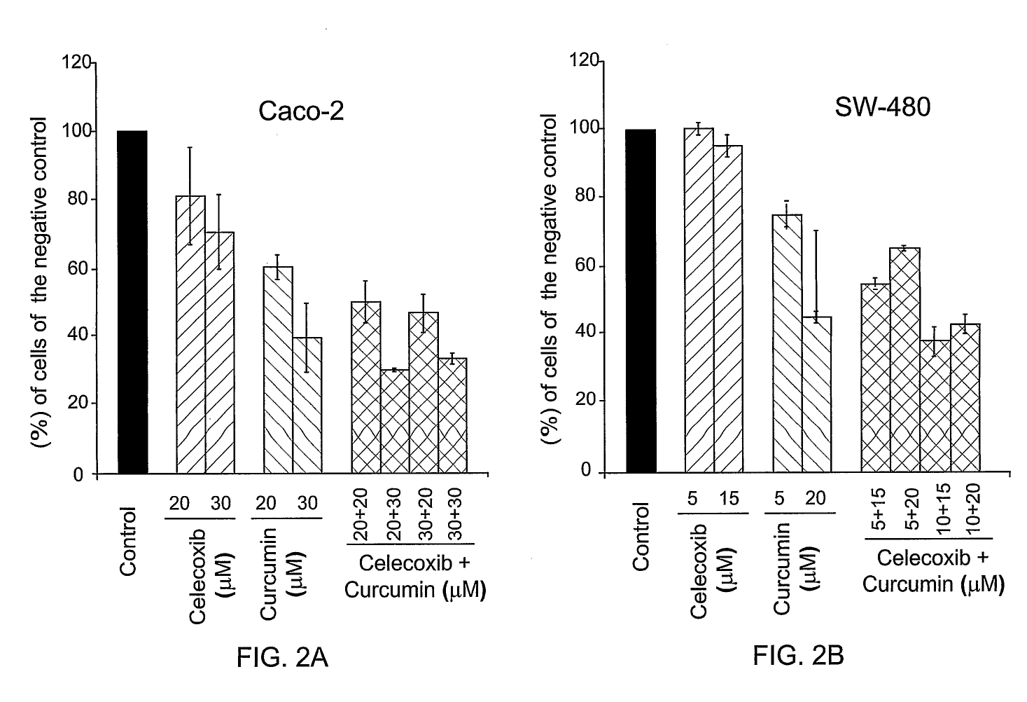

FIGS. 2A and 2B depict the effect of celecoxib and curcumin on cell growth of human colon carcinoma cell line Caco-2 (FIG. 2A) and SW480 (FIG. 2B).

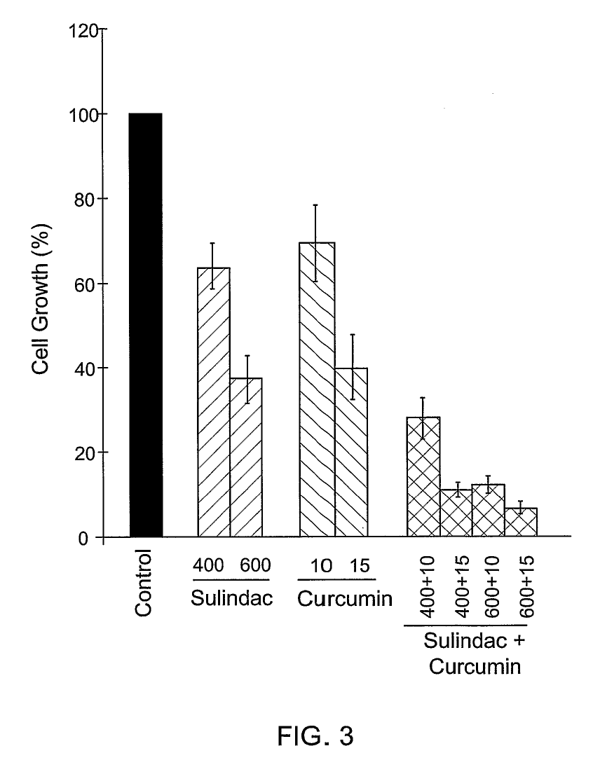

FIG. 3 depicts the effect of sulindac and curcumin on cell growth of the human colon carcinoma cell line HT-29.

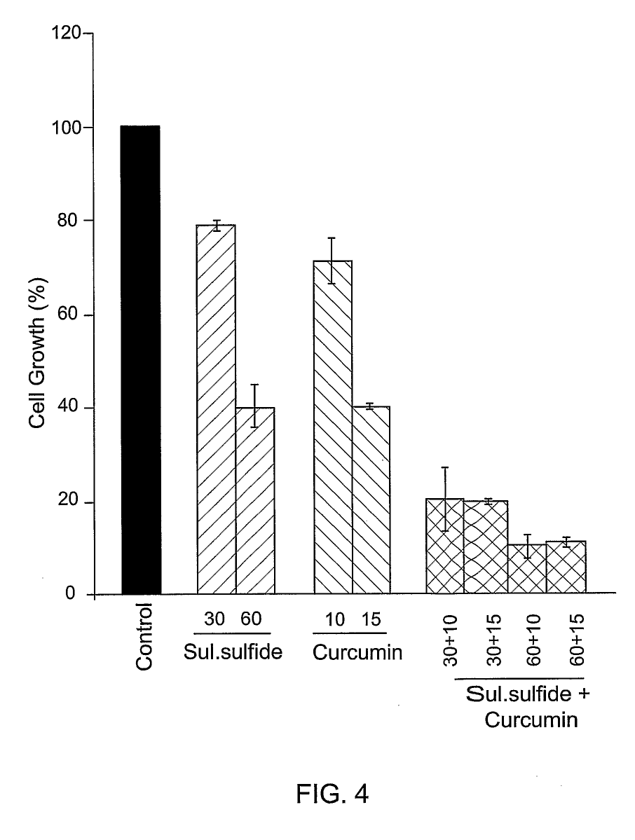

FIG. 4 depicts the effect of sulindac sulfide and curcumin on cell growth of the human colon carcinoma cell line HT-29.

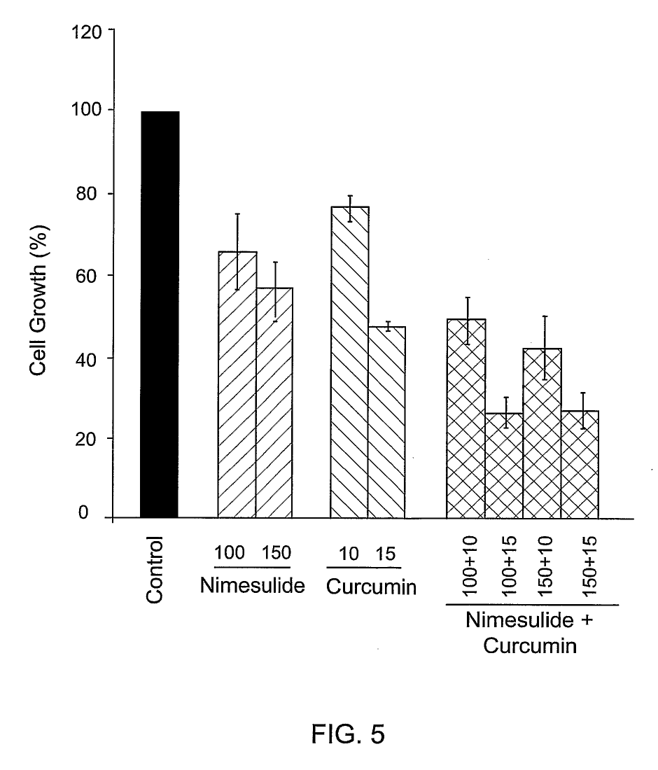

FIG. 5 depicts the effect of nimesulide and curcumin on cell growth of the human colon carcinoma cell line HT-29.

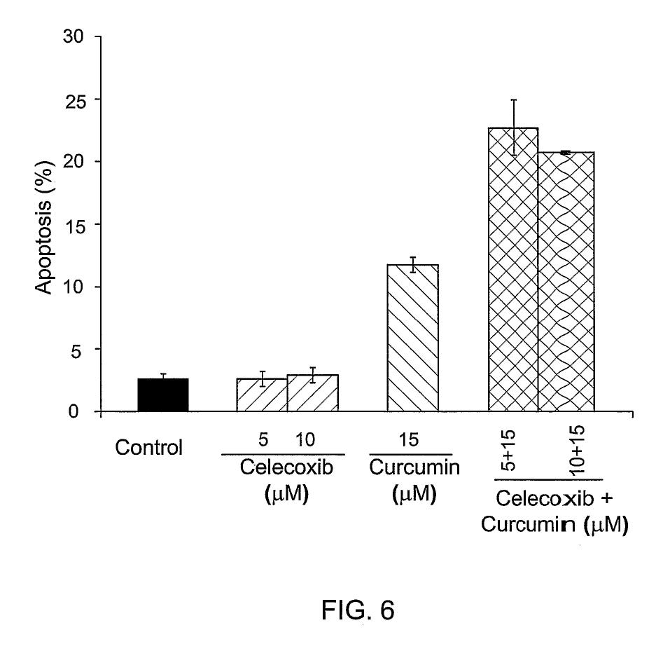

FIG. 6 depicts the effect of celecoxib and curcumin on HT-29 cell line apoptosis.

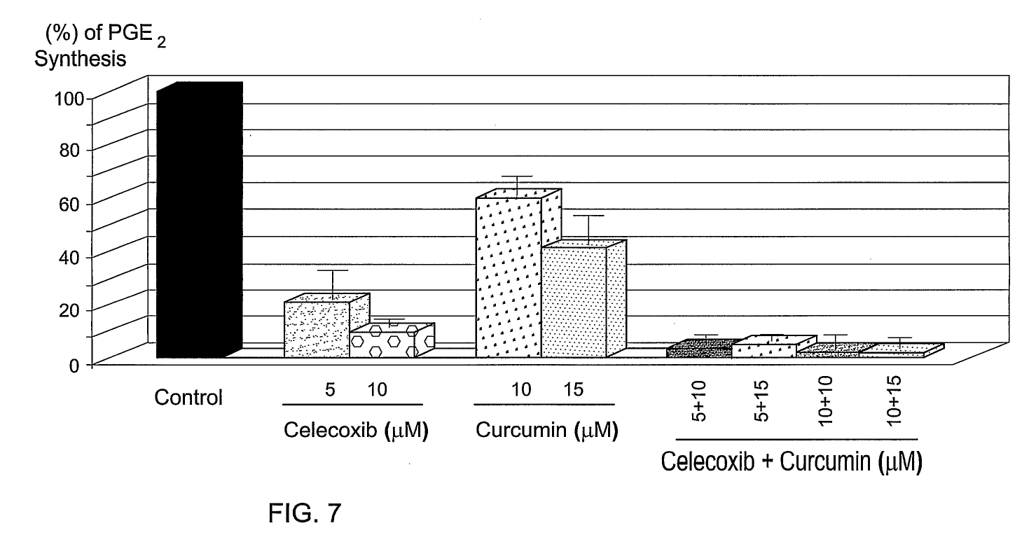

FIG. 7 depicts the effect of celecoxib and curcumin on PGE₂ synthesis in HT29 cell line.

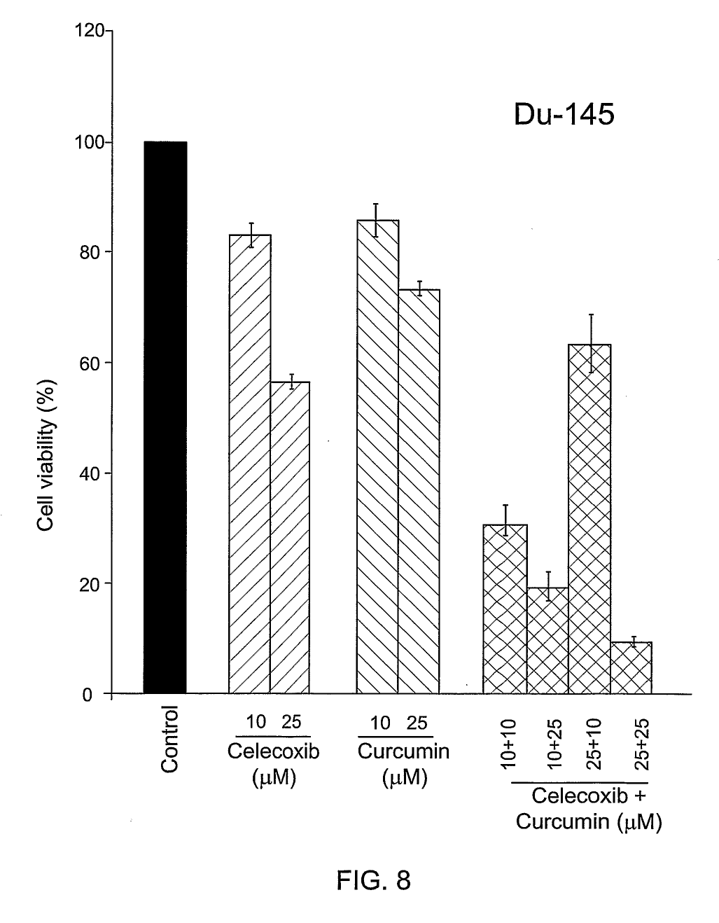

FIG. 8 depicts the effect of celecoxib and curcumin on cell growth of human prostate cancer cell line Du-145.

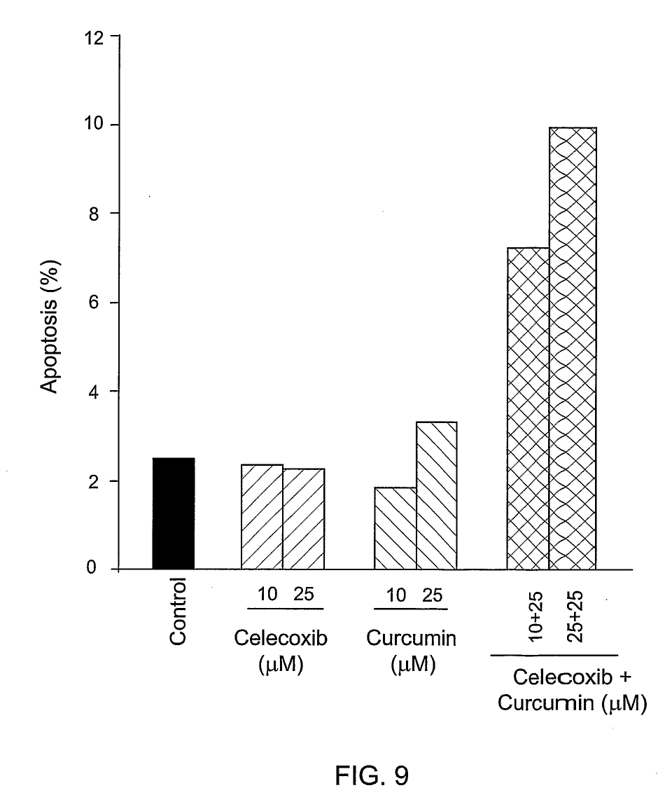

FIG. 9 depicts the effect of celecoxib and curcumin on apoptosis of Du-145 cell line.

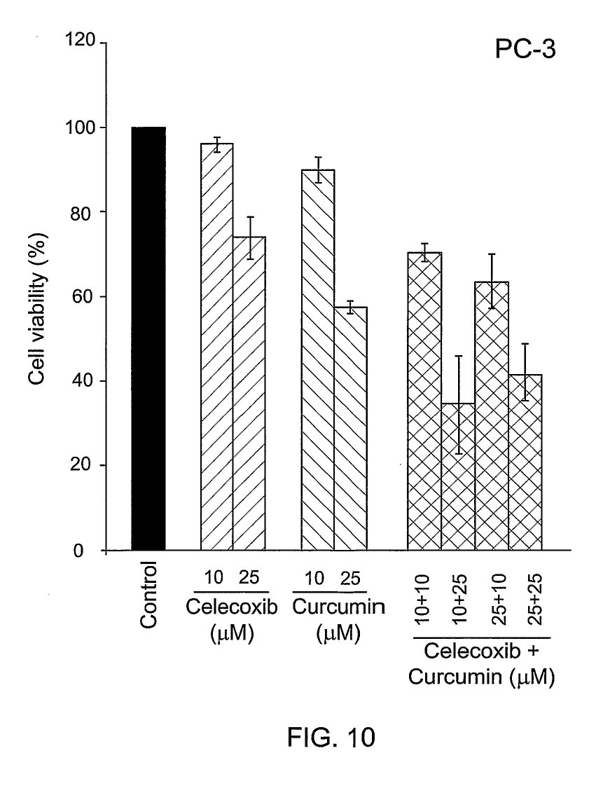

FIG. 10 depicts the effect of celecoxib and curcumin on cell growth of human prostate cancer cell line PC-3.

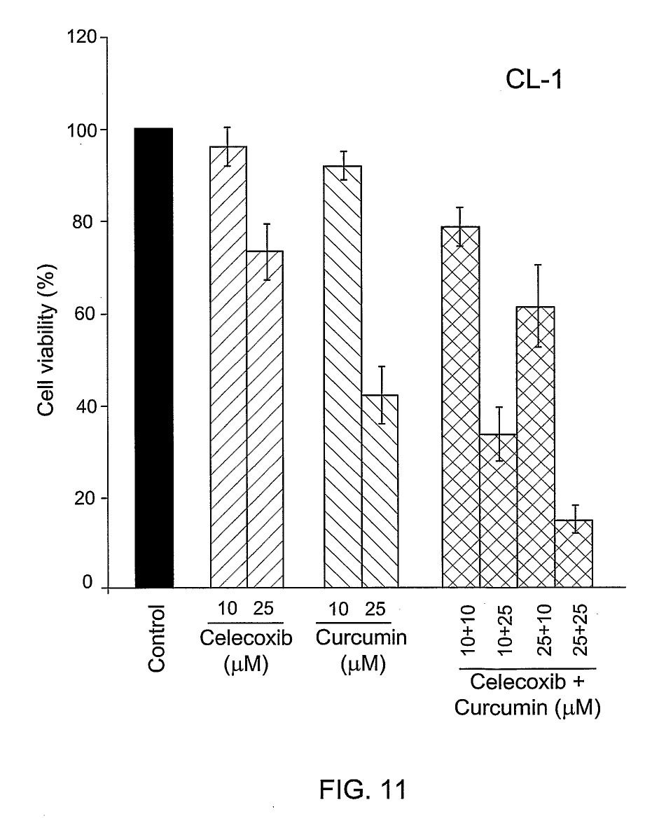

FIG. 11 depicts the effect of celecoxib and curcumin on cell growth of human prostate cancer cell line Cl-1.

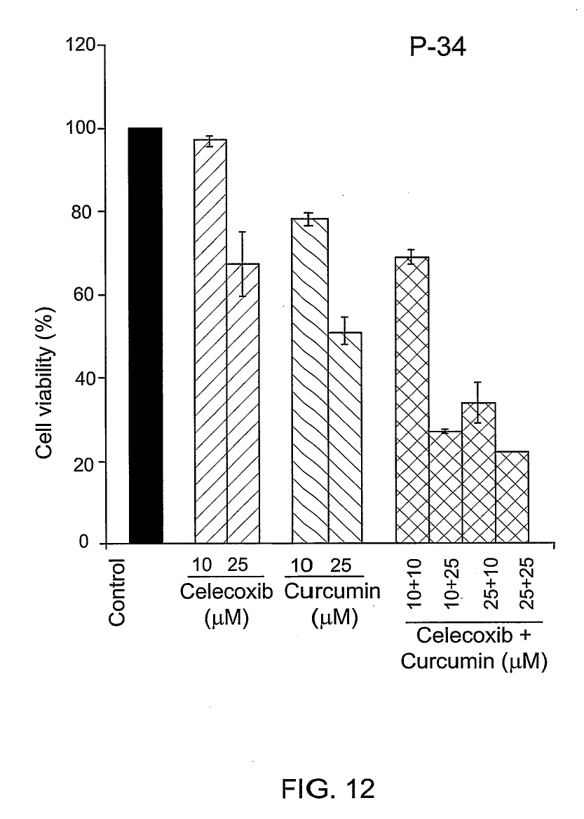

FIG. 12 depicts the effect of celecoxib and curcumin on cell growth of human pancreas cancer cell line P-34.

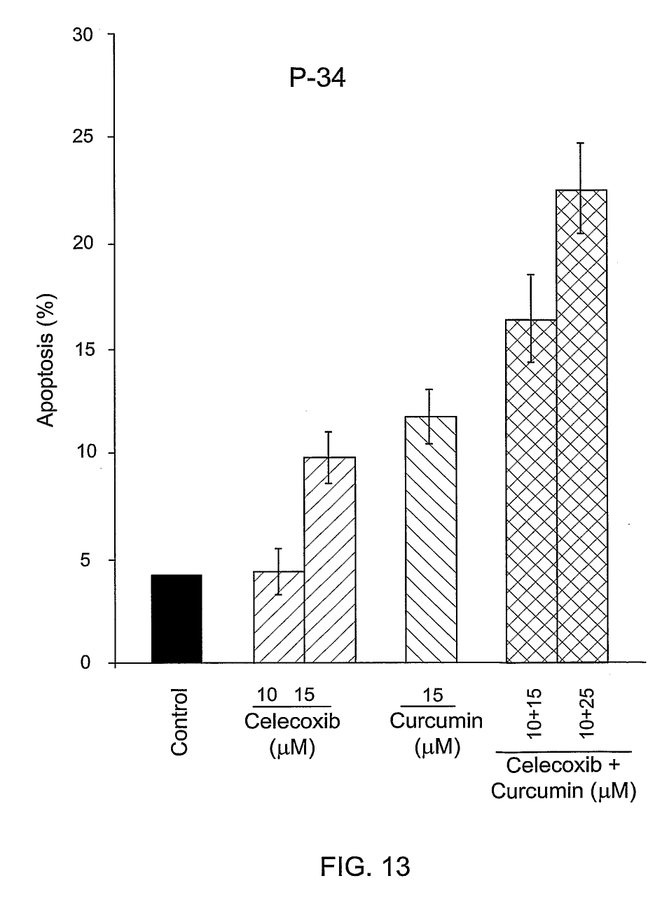

FIG. 13 depicts the effect of celecoxib and curcumin on apoptosis of P-34 cell line.

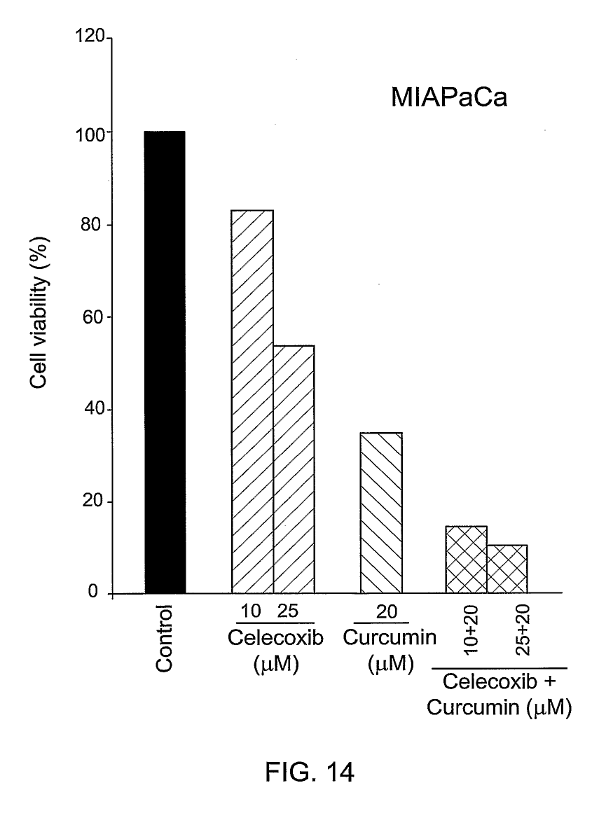

FIG. 14 depicts the effect of celecoxib and curcumin on cell growth of human pancreas cancer cell line MIAPaCa.

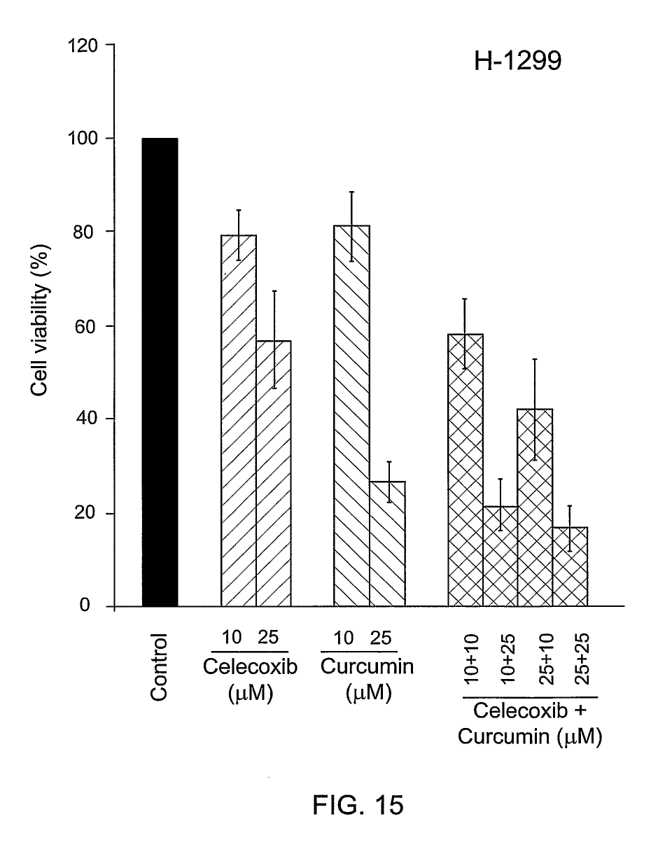

FIG. 15 depicts the effect of celecoxib and curcumin on cell growth of human lung cancer cell line H-1299.

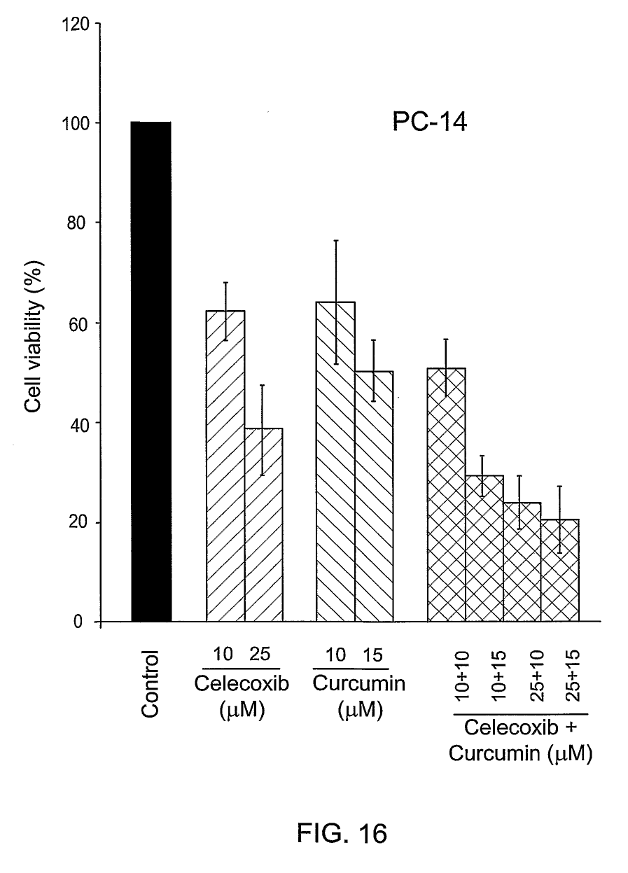

FIG. 16 depicts the effect of celecoxib and curcumin on cell growth of human lung cancer cell line PC-14.

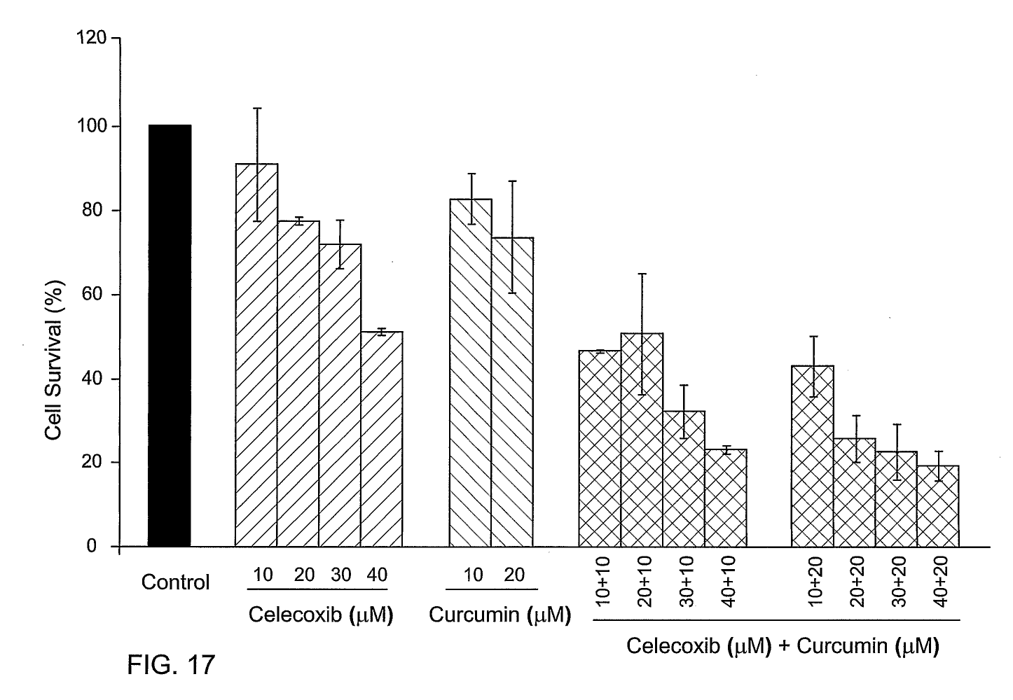

FIG. 17 depicts the effect of celecoxib and curcumin on cell growth of human RASF cells.

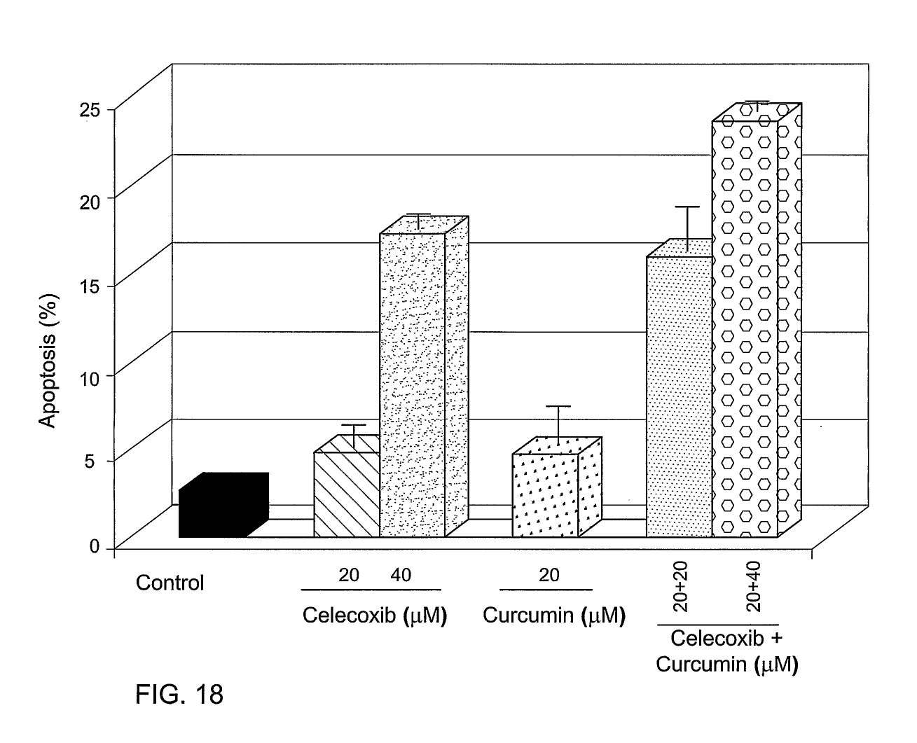

FIG. 18 depicts the effect of celecoxib and curcumin on apoptosis of RASF cell line.

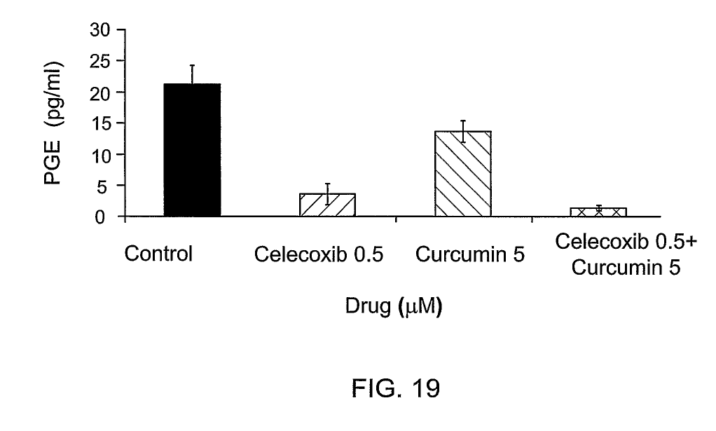

FIG. 19 depicts the effect of celecoxib and curcumin on PGE₂ synthesis in RASF cell line.

### MATERIALS AND METHODS

Reagents and Chemicals

Curcumin was purchased from Merck (White House, N.J., USA). Celecoxib was provided by Pfizer (New York, N.Y., USA). Sulindac and Sulindac Sulfide were provided by Cell Pathways Inc. (Horsham, Pa., USA). All other reagents with the highest purity were purchased from Sigma Chemical Co. (St. Louis, Mo., USA).

I. Cancer Study

1. Cancer Cell Culture

The growth inhibition of celecoxib, sulindac, sulindac, sulindac sulfide, curcumin or an analogue or derivatives thereof such as demethoxycurcumin and bisdemethoxycurcumin, and combination of curcumin with celecoxib, with sulindac or with sulindac sulfide was tested on the following cell lines:

1. Human colon adenocarcinoma cell line HT-29 obtained from the American Type Culture Collection (ATCC) and

2. Human colon adenocarcinoma cell line SW-480 obtained from the American Type Culture Collection (ATCC) and

3. Human colon adenocarcinoma cell line Caco-2 obtained from the American Type Culture Collection (ATCC) and

4. Normal enterocytes derived from the rat Ileum (IEC-18 cells) and transformed by c-K-ras oncogen (IEC-18-K-ras cells) were also used (Arber, N. et al., Oncogene, 12:1903-1908, 1996) and

5. Human prostate cancer cell line Cl-1 obtained from the American Type Culture Collection (ATCC) and

6. Human prostate cancer cell line PC-3 obtained from the American Type Culture Collection (ATCC) and

7. Human prostate cancer cell line Du-145 obtained from the American Type Culture Collection (ATCC) and

8. Human pancreas cancer cell line P-34 obtained from Dr. Star, Sourasky Medical Center, Tel-Aviv, Israel and

9. Human pancreas cancer cell line MRPaCa obtained from the American Type Culture Collection (ATCC) and

11. Human pancreas cancer cell line Panc-1 obtained from the American Type Culture Collection (ATCC) and

12. Human lung cancer cell line PC-14 obtained from Dr. Fidler, M.D., Anderson Cancer Center, Houston, Tex. and

13. Human lung cancer cell line H1299 obtained from the American Type Culture Collection (ATCC) and

The different cell lines were grown and maintained in Dulbecco's modified Eagle's medium (DMEM, Biological industries, Israel) supplemented with 5% fetal calf serum (FCS), penicillin and 1% streptomycin at 37° C., in an atmosphere of 95% oxygen and 5% CO₂ (herein referred to as “full medium”).

2. Assays for Growth Inhibition of Cancer Cells

Cells were plated in duplicate at a density of 3×10⁴ in 12-well plates containing 1 mL of full medium. Celecoxib, curcumin or 0.1% dimethyl sulfoxide (DMSO, the drug vehicle) were added to the culture medium 24 hours after plating. The numbers of viable cells after incubation with these compounds for 72 hours was determined in duplicates using a Coulter counter. All experiments were repeated at least three times and gave similar results. The different combinations of celecoxib and curcumin were used similarly and the viable cells were similarly counted.

The same procedure was repeated for curcumin and sulindac and for curcumin and sulindac sulfide.

3. Assay for Cell Viability

HT-29 cells (5×103 per well) were seeded in 96-well plastic plates and incubated at 37° C. in full medium containing the test drugs. After 48 and 72 hours, cell viability was assessed by the ability of metabolically active cells to reduce tetrazolium salt (XTT) to colored formazan compounds. The absorbance of the samples was measured with an ELISA reader (wavelength 450 nm, reference wavelength 630 nm). Each measurement was performed in triplicate. The data are mean values from three different experiments.

4. Apoptosis Assays

Apoptosis was determined by two independent methods:

A. Flow-Cytometric Analysis

HT-29 cells were plated at a density of 5×106 per 10-cm dish with test drugs at selected concentrations. The adherent and non-adherent cells were collected during exponential growth of the cells and counted. A total of 1-2×106 cells were washed in phosphate-buffered saline (PBS) and the pellet was fixed in 3 ml ethanol for 1 hour at 4° C. The cells were pelleted and re-suspended in 1 ml PBS and incubated for 30 minutes with 0.64 mg/ml RNAse at 37° C. They were stained with 45 μg/ml PI at least 1 hour before analysis by flow cytometry using a standard protocol for cell cycle distribution and cell size.

Necrotic cells were excluded by counting cells following staining with trypan blue before fixation. All experiments were done three times and gave similar results. Data acquisition was performed on a FACScan and analyzed by CellQuest software (Becton Dickinson Immunocytometry Systems, San Jose, Calif., USA). All fluorescence and laser light scatter measurements were made with linear signal processing electronics. Data for at least 10,000 cells were collected for each data file.

B. Fluorescence Microscopy

Apoptotic cells were detected by nuclear morphologic changes using PI staining. Cells were washed twice with PBS and fixed for 15 minutes at room temperature with 4% paraformaldehyde in PBS. The fixative was removed by aspiration, and the monolayer was washed twice in PBS. DNA was incubated with 0.15 mg/ml RNAse for 15 minutes and stained with 5 μg/mL PI at room temperature. Excess PI stain was removed, and the monolayer was thoroughly washed with PBS. The cover slip was mounted with Glycerol. The stained nuclei were viewed at ×63 using a Lieca TCS SP2 confocal microscope (Lieca Microsystems, Wetzler, Germany).

5. Protein Extraction and Western Blotting

Exponentially growing cells were collected and washed three times in ice-cold PBS as described earlier. The cell pellets were re-suspended in lysis buffer (20 mM Tris-HCI pH7.4, 2 mM EDTA, 6 mM 6-mercaptoethanol, 1% NP-40, 0.1% SDS and 10 mM NaF, plus the protease inhibitors leupeptin 10 μg/ml, aprotinin 10 μg/ml and 0.1 mM phenylmethylsulfonylfluoride). The protein concentration of each sample was estimated using the Bio-Rad protein assay (Bio-Rad Laboratories, Calif., USA). For western blotting, samples containing 50 μg of total cell lysate were loaded onto a 10% SDS-polyacrylamide gel and subjected to electrophoresis. Proteins were transferred to “Hybond-C” membranes (Amersham, Arlington Heights, Ill., USA) in transfer buffer (25 mM Tris, 190 mM glycine, 20% methanol), using a Trans Blot transfer apparatus at 70 mA for 12-18 hours at room temperature. The membranes were blocked with blocking buffer (PBS/0.2% Tween 20/0.5% gelatin) for 1 hour at room temperature and subsequently washed three times for five minutes in washing buffer (PBS/0.05% Tween-20). The membranes were incubated with monoclonal human anti-COX-1, anti-COX-2, Actin antibodies for 1 hour at room temperature. The membranes were washed as described above and incubated with anti-goat secondary antibodies (1:2000) for one hour at room temperature. Additional washes were carried out as described previously, and immune detection was performed using the ECL Western blotting detection system (Amersham, Arlington Heights, Ill., USA).

6. Reverse Transcriptase PCR

HT-29 cells were treated with the selected drugs as indicated. The mRNA was extracted, and equal amounts were transcribed to cDNA from a kit (Promega, CITY, USA). The cDNA was taken from samples at various times and used as the DNA templates for PCR. Primers used for COX-2 were:

5′- TTC AAA TGA GAT TGT GGG AAA AT -3′
and
5′- AGA TCA TCT CTG CCT GAG TAT CTT-3′.

GAPDH was used to ensure equal loading. Two milliliters were used as the template and 1 milliliter of each primer was used for each cDNA sample. The samples went through 30 rounds of PCR. They were separated on 1% agarose gel and visualized by ethidium bromide.
7. Measurement of PGE₂ Concentration

PGE₂ concentration in the medium, as released by the cells, was determined by a commercially available PGE₂-specific enzyme-linked immunoassay (R&D Biosystems, Abingdon, UK) according to the manufacturer's protocol.

8. Statistical Analysis

The results were measured as mean±SD. To evaluate the difference between treatment with each of the drugs and treatment with their combination, the one-way ANOVA test was performed using an SPSS software package (SPSS Inc., Chicago, Ill., USA). Statistical significance (P<0.05) was established by the post hoc Tukey's pair wise comparison.

9. RESULTS of the Cancer Study

Curcumin augments celecoxib's growth-inhibitory effect in human cancer cell lines in vitro. This observed effect is clinically important, as it can be achieved in the serum of patients receiving standard anti-inflammatory doses of celecoxib. Our current results demonstrate that in the presence of low concentrations of curcumin (10-15 μM), a physiological concentration of celecoxib (5 μM) is sufficient to inhibit cell growth by inhibiting proliferation and inducing apoptosis, by COX-2 and non-COX-2 pathways. This effect is similar to that achieved with a 10-fold higher concentration of celecoxib (50 μM) when administered alone. The clinical importance of this effect lies in the fact that it can be achieved in the serum of patients treated with a standard anti-inflammatory (200-400 mg) or anti-neoplastic (400-800 mg) doses of celecoxib. This paves the way for a novel strategy to prevent and treat cancers of various types, given that this approach will involve a regimen of a low profile of side effects. A synergistic effect is also observed in HT-29 and IEC18-K-ras cells that express high levels of COX-2. Only the combined modality regiment reduced the level of COX-2 mRNA and almost entirely diminishing PGE₂ production. At the same time a significant additive growth inhibition was observed in cancer cell lines which express low or no COX-2 activity (e.g. Caco-2 and SW-480).

It should be noted, that while the examples given herein demonstrate the use of celecoxib in combination with curcumin, these examples should be considered as non-limiting. Further experiments were conducted using other NSAIDs such as sulindac, sulindac sulfide, nimesulide and others as listed hereinabove.

A. NSAID Drugs and Curcumin Inhibited Cell Growth

The effect of celecoxib and curcumin on cell growth of three human colon carcinoma cell lines (HT-29, SW480 and Caco-2) and the c-K-ras-transformed rat intestinal epithelial cell line (IEC18-ras, designated in the accompanying figures as R1) was assessed alone and in combination. An inhibitory effect of curcumin or celecoxib on cell growth of the cell lines was found to be dose dependent.

In HT-29 cells that express high levels of COX-2 protein, treatment with either celecoxib (5-10 μM) or curcumin (10-15 μM) resulted in a minor (25-30%) inhibition of cell growth. When celecoxib and curcumin were combined (5 μM and 10 μM, respectively), there was 80-90% reduction in cell number (FIG. 1A). This effect was similar to that exerted by a 10-fold higher concentration of celecoxib (50 μM) alone (not shown). A similar effect was observed in another cell line (IEC18-K-ras, designated as R1) that expressed a high level of COX-2 (FIG. 1B). A weaker effect shown in FIGS. 2A and 2B was observed in the Caco-2 cells that produce low levels of COX-2, and in SW-480 cells that do not express COX-2. Cell viability assays confined these results (data not shown).

A summary of the growth inhibitory effect is shown in Table 1 for the combination in various cell lines. HT-29 cells, in which the maximal effect was observed, were chosen for further studies. These cells express high levels of COX-1 and COX-2 mRNAs and proteins.

TABLE 1

| Cell | Combined Effect | COX-2 Expression |
|---|---|---|
| HT-29 | ++ | ++ |
| IEC18-ras | ++ | ++ |
| Caco-2 | + | + |
| SW-480 | +/− | − |

Summary of the combined effects, wherein for the column of Combined Effect:
(++) refers to >5 fold effect;
(+) refers to 2-5 fold effect;
(+/−) refers to 1-2 fold effect;
(−) refers to <1 fold effect and for the COX-2
Expression column:
(++) refers to high level of expression;
(+) low level of expression and (−) no expression.

Similar results were obtained for curcumin and sulindac (FIG. 3), for curcumin and sulindac sulfide (FIG. 4) and for curcumin and nimesulide (FIG. 5).

B. Celecoxib and Curcumin Induced Apoptosis

The extent of apoptosis was assessed by flow cytometry analysis following 72 hours exposure of HT-29 cells to the different treatments. The combination of is celecoxib (5 μM) and curcumin (15 μM) significantly increased the percentage of cells with sub-diploid DNA content, the hallmark of apoptosis (FIG. 6), compared to treatment with each drug alone.

Drug-treated HT-29 cells were examined for morphological evidence of apoptosis using fluorescence microscopy, following PI staining. Typical apoptotic features of chromatin condensation and nuclear fragmentation were seen in the treated cells only when they were exposed to both drugs.

C. The Combination Therapy Involved Down-Regulation of COX-2 Activity and mRNA Expression

To determine whether the combination effect involves down-regulation of COX-2 synthesis, HT-29 cells were treated with celecoxib, curcumin and their combination at selected concentrations. The mRNA was isolated and probed with COX-2 cDNA. Each compound alone had no effect on the expression of COX-2 mRNA, whereas the two drugs significantly down-regulated COX-2 mRNA expression.

The question of whether COX-2 inhibition is required for growth inhibition was studied as well. PGE₂ synthesis was evaluated in HT-29 cells as a measure of COX-2 activity. Treatment with low concentrations of celecoxib (5 μM) resulted in 80% inhibition of PGE₂ production. Curcumin (10-15 μM) had a dose-dependent effect on PGE₂ synthesis. The addition of curcumin to celecoxib almost totally (>95%) diminished PGE₂ synthesis (FIG. 7).

As shown in FIGS. 8 to 16, the composition of the present invention was also effective respectful of prostate, pancreas and lung cancers.

II. Inflammatory Study

1. Culture of Osteorthritis Synovial Fiborblasts (RASFs)

RASFs that had been prepared from small pieces (2 mm in diameter) of human synovial tissue were grown after trypsinization in monolayers in tissue culture flasks, as known to a person skilled in the art. The synovial tissue specimens were obtained during total knee replacement surgery in patients diagnosed as having rheumatoid arthritis according to the revised criteria of the American College of Rheumatology. Synovial tissue was digested for 2 hours with 0.2% (weight/volume) bacterial collagenase and then suspended in Dulbecco's modified Eagle's medium (DMEM, Biological Industries, Israel) with 10% (volume/volume) fetal calf serum (FCS), 100 units/ml penicillin, and 100 mg/ml streptomycin. The cells were incubated at 37° C. in 5% CO₂ for 3-5 days, after which the non-adherent cells were removed. Fibroblast-like adherent cells from the first or second passage were used as RASFs.

2. Cell Viability Assay

RASFs (1.5×10⁴/well) were incubated at 37° C. in 96-well plastic plates with test drugs in DMEM containing 10% FCS in an atmosphere of 5% CO₂ (fmal dimethyl sulfoxide concentration 0.1%). After 72 hours, cell viability was assessed by the ability of metabolically active cells to reduce tetrazolium salt (XTT) to colored formazan compounds. The absorbance of the samples was measured with a specific enzyme-linked immunoassay (ELISA) reader (wavelength 450 nm; reference wavelength 630 nm). Each measurement was done in triplicate. All experiments were repeated at least three times.

4. Flow-Cytometric Analysis

RASF cells were plated at a density of 5×10⁶ per 10 cm dish with the various test drugs at selected concentrations. The adherent and non-adherent cells were collected during exponential growth of the cells and counted. A total of 1-2×106 cells were washed in phosphate-buffered saline (PBS) and the pellet was fixed in 3 ml ethanol for 1 hour at 4° C. The cells were pelleted and resuspended in 1 ml PBS and incubated for 30 minutes with 0.64 mg/ml RNAse at 37° C. The cells were stained with 45 μg/ml propidium iodide for at least 1 hour before analysis by flow cytometry using a standard protocol for cell cycle distribution and cell size.

Necrotic cells were counted using trypan blue before fixation. All experiments were done three times. Data acquisition was performed on a FACScan and analyzed using CellQuest software (Becton Dickinson Immunocytometry Systems, San Jose, Calif., USA). All fluorescence and laser light scatter measurements were made with linear signal processing electronics. Data for at least 10,000 cells were collected for each data file.

5. Protein Extraction and Western Blotting

Exponentially growing cells were collected and washed three times in ice-cold PBS as described earlier. The cell pellets were re-suspended in lysis buffer (20 mM Tris-HCI pH7.4, 2 mM EDTA, 6 mM 6-mercaptoethanol, 1% NP-40, 0.1% SDS and 10 mM NaF, plus the protease inhibitors leupeptin 10 μg/ml, aprotinin 10 μg/ml and 0.1 mM phenylmethylsulfonylfluoride). The protein concentration of each sample was estimated using the Bio-Rad protein assay (Bio-Rad Laboratories, CA, USA). For western blotting, samples containing 50 μg of total cell lysate were loaded onto a 10% SDS-polyacrylamide gel and subjected to electrophoresis. Proteins were transferred to “Hybond-C” membranes (Amersham, Arlington Heights, Ill., USA) in transfer buffer (25 mM Tris, 190 mM glycine, 20% methanol), using a Trans Blot transfer apparatus at 70 mA for 12-18 hours at room temperature. The membranes were blocked with blocking buffer (PBS/0.2% Tween 20/0.5% gelatin) for 1 hour at room temperature and subsequently washed three times for five minutes in washing buffer (PBS/0.05% Tween-20). The membranes were incubated with monoclonal human anti-COX-1, anti-COX-2 and Actin antibodies for 1 hour at room temperature. The membranes were washed as described above and incubated with anti-goat, secondary antibodies (1:2000) for one hour at room temperature. Additional washes were carried out as described previously, and immune detection was performed using the ECL Western blotting detection system (Amersham, Arlington Heights, Ill., USA).

6. Measurement of PGE₂ Concentration

PGE₂ concentration in the medium, as released by the RASF cells, was determined by a commercially available PGE₂-specific ELISA (R&D biosystems, Abingdon, UK) according to the manufacturer's protocol.

7. Measurement of PGE₂ Levels

PGE₂ concentration released by the cells was determined by a PGE₂-specific enzyme-linked immunoassay (R&D biosystems, Abingdon, UK) according to the manufacturer's protocol.

9. Statistical Analysis

The results were assessed as mean±SD. The difference between treatments with each of the drugs and with their combination was evaluated by the one-way analysis of variance test using SPSS software package (SPSS Inc., Chicago, Ill., USA). Statistical significance (P<0.05) was established by post hoc Tukey's pairwise comparison.

To determine if the combination was additive or synergistic, we used the analysis of Loewe (Loewe S., et al., Arzneim-Forsch 1953, 3:285-320, and also Aghi M, et al., J. Natl. Cancer Inst. 1998, 90(5):370-80), which is used where the effects of two drugs are mutually exclusive (i.e., the drugs possess similar modes of action, such as cox-2 inhibition). In brief, in an isobologram, the x and y axes represent doses of drugs 1 and 2. A straight line is then drawn for any Fa (fraction of cells effected by the treatment) value of interest representing doses of drug 1+drug 2 that would be required to achieve the given Fa value if the two drugs were additive. The observed experimental concentrations at which combined treatment generated the given Fa value are plotted in the isobologram: synergism is indicated if these points lie to the lower left of the curve/straight line, defining additivity at that Fa value; additivity is indicated if the experimental points lie on the curve/straight line; antagonism is indicated if they lie to the upper right of the curve/straight line.

10. Results of the Inflammation Studies

Combinatorial therapy of rheumatoid arthritis is based on the multifactorial nature of chronic inflammation. The concurrent use of drugs with different mechanisms of action or pharmacokinetics may be more effective and less toxic than each of the monotherapeutic regimens alone. Furthermore, toxicity can be reduced, particularly when the treatment is comprised of a commonly used dietary factor such as curcumin.

Synovial fibroblasts secrete mediators of inflammation and joint destruction and are recognized as important players to the pathogenesis of osteoarthritis. Therefore, induction of apoptosis of these cells to induce a long-term remission is an attractive therapeutic goal.

Without wishing to be bound by theory, the data presented thus far suggests that the combined therapy involved both COX-2 and non-COX-2 mechanisms. The addition of curcumin (5 μM) to celecoxib (0.5 μM) augmented its effect by almost totally diminishing PGE₂ production (>95%), indicating that curcumin augments the NSAID inhibitory effect on COX-2 activity. Potentiation of the effect of celecoxib was, however, also seen in concentrations >10 μM in which the NSAID alone almost totally inhibited PGE₂ production in RASF cells (data not shown).

This data reveals that curcumin augments the inhibition of RASF cell growth and enhances the induction of apoptosis by celecoxib. The synergistic or additive effect observed may be mediated through a mechanism that involves inhibition of COX-2 activity. This enables the use of celecoxib at lower and safer concentrations and may pave the way for a novel combinatorial treatment of inflammatory diseases or disorders.

It should be noted, that while the examples given herein demonstrate the use of celecoxib in combination with curcumin, these examples should be considered as non-limiting. Further experiments were conducted using other NSAIDs such as sulindac, sulindac sulfide, nimesulide and others as listed hereinabove.

A. Celecoxib and Curcumin Inhibited Cell Growth

The effect of celecoxib and curcumin on cell growth of RASFs was assessed alone and in combination using the XTT assay. Treatment with celecoxib (IC₅₀=40 μM) or curcumin (IC₅₀=35 μM) inhibited cell growth in a dose-dependent matter. The addition of curcumin (10-20 μM) potentiated the effect of celecoxib on cell growth (FIG. 17).

B. Curcumin Augmented the Effect of Celecoxib on the Induction of Apoptosis

To investigate whether the effect combining the drugs had on cell growth could result from its effect on apoptosis, we examined the effect of the different treatments on the percentage of cells with sub-diploid DNA content using the flow cytometry analysis.

Celecoxib at concentrations, of 20-40 μM demonstrated a moderate effect (4.8-19.2%) on cell apoptosis (FIG. 18). Curcumin at a concentration of 20 μM caused a minor effect (4.7%), but it demonstrated a significantly enhanced effect on cell apoptosis when it was applied together with celecoxib (20-40 μM).

C. Curcumin Potentiates the Effect of Celecoxib on PGE₂ Synthesis

In order to evaluate whether the inhibition of cell growth by the combination of drugs was correlated with COX-2 inhibition, the effect of various treatments on COX-2 activity was assessed: 0.5 μM of celecoxib inhibited PGE₂ production by 80% (FIG. 19) and the addition of curcumin (5 μM) to celecoxib almost totally diminished (>95%) PGE₂ synthesis.

III. In Vivo Studies

1. Animals Models for Cancer:

In all the experiments described below, sets of normal and transformed cells that were developed during the investigations leading to the present invention will be used in order to produce tumors at various sites of outbred male germfree CD(SD)GN mice.

The mice are randomly assigned to one of four treatment groups (eight animals per group). The control group is fed regular Purina chow. The other groups receive a standard diet supplemented with either celecoxib, curcumin or their combination. Food consumption, weight change, general health status and signs and symptoms of toxicity are measured twice weekly during the experiment and at the end of the study.

Necropsy includes gross examination of all internal organs including the lungs, heart, stomach, spleen, kidneys and liver. Each tumor diameter is measured using a micrometer caliper. Tumor volume, in mm³, will be calculated using the formula V=4/3πr³, or by using the ovoid volume as most tumors grown in animals are ovoid in shape. Both the number of mice with tumors (incidence) and the volume of tumors per mice (tumor burden) are measured. At the termination of the experiment, animals are sacrificed following sodium pentothal anesthesia and cervical dislocation.

A. The Protective Effects of Combined Celecoxib and Curcumin on the Induction of Subcutaneous Tumors

Subcutaneous tumors are produced by injecting 0.25 ml of viable tumor cells (2.5×106) into two flanks of each animal. The control and experimental diets are continued for up to 10 weeks, at which point the experiment is terminated. The animals are palpated twice weekly to detect the presence and location of subcutaneous tumors. The time of appearance of the first tumor (latency period) and its size is recorded. The mice are fed with the drugs one week before the tumor induction.

B. The Protective Effects of Celecoxib on the Induction of Colorectal Cancer

For induction of colon cancer to produce colonic tumors in the cecum, each mouse is anesthetized and its abdomen is prepared for sterile surgery. Then, 0.1 ml of viable tumor cells (1×106) is injected into the cecal wall from the serosal side. The serosal surface, at the injection site, is dabbed gently with sterile gauze dampened with 95% ethanol to kill tumor cells that may have escaped. The abdominal contents are returned to the abdominal cavity and surgical clips are used to close the incision. The mice are then allowed to recover and the surgical clips are removed after the laparotomy incision has completely healed. After 3-4 weeks, the cecum and the abdominal cavity are examined for the presence of macroscopic tumors. Cecal tumors are grossly evident by 2 weeks. The optimum timing for the development of tumors in the cecum is 14-21 days, depending on the cell type. The mice are fed with the drug one week before tumor induction.

C. Does Celecoxib and Curcumin Protect Against Metastasis Formation—The Spleen Model

In this model for metastatic liver formation to produce liver metastasis, the mice are placed in the supine position, anesthetized, and their abdomen prepared for sterile surgery. Under sterile conditions, a flank incision is made, the spleen exposed and 0.05 ml (0.5×106) of viable tumor cells are injected into the splenic sub-capsule. The abdominal contents are returned to the abdominal cavity and surgical clips are used to close the incision. The mice are allowed to recover and the surgical clips are removed after laparotomy incision has completely healed. The abdominal organs are examined for the presence of macroscopic tumors and for liver metastases in particular. Metastatic nodules in the liver are detectable macroscopically and confirmable microscopically after H&E staining. The percentage of mice with hepatic metastasis is recorded. The optimum timing for the development of liver metastasis is 18-21 days, depending on the cell type. The mice are with the drug one week prior to tumor induction.

Drug levels are evaluated in terminal-bleed plasma samples taken from all mice after they were sacrificed. Blood is collected in heparin red tubes and the plasma is separated and frozen at −70° C., before preparation for BPLC analysis. 100 μl of plasma is mixed with an equal volume of acetonitrile, centrifuged at 10,000×g for 15 min, and a 25-μl aliquot of the supernatant is analyzed for example by reverse-phase HPLC separation, for example on a EP1090 system (Hewlett-Packard, Palo Alto, Calif.) with an Eclipse XDB-C18 rapid resolution column (75×4.6 mm, 3.5 μm; Hewlett Packard). Drug concentrations are determined, in comparison with standard curves and separated under identical conditions.

2. Animals Models for Arthritis:

Two Animal models of arthritis will be studied:

A. Collagen induced arthritis (CIA)—is an animal model for rheumatoid arthritis (RA) that is widely used to address questions of disease pathogenesis and validate therapeutic targets and agents (Method Mol Med). DBA/1 mice are immunized with authologous or heterologous type II collagen in adjuvant. As in RA, there is a robust T and B cell response, accompany with prolifemiceive synovitis with infiltmiceion of polymorphonuclear and mononuclear.

The effect of celecoxib, curcumin and their combination on DBA1 male mice, injected with collagen type II, is assessed. The Mice are assigned to one of four treatment groups (eight animals per group). The control group is fed regular diet. The other groups receive a standard diet supplemented with celecoxib, curcumin or celecoxib and curcumin combined.

Collagen induced arthritis features is evaluated by such anti-inflammatory parameters as characterization of the site of lesion, lymphokine secretion profile (TNF-a, IL-1b, IL-6, IL-10, INFg) in the animal serum.

B. Pristane-induced arthritis (PIA)—a chronic disease that is induced by two intraperitoneal injections of pristane, a synthetic adjuvant oil. The animals develop arthritis with late onset of clinical disease. There is persistent joint inflammation, synovial hyperplasia, inflammatory cell infiltmiceion and presence of rheumatoid factor. Characterization of the effect of celecoxib and curcumin synergism on BALB/c mice, injected with the synthetic adjuvant oil, pristine is evaluated. The Mice are randomly assigned to one of four treatment groups (eight animals per group). The control group is fed regular diet. The other groups receive a standard diet supplemented with celecoxib, curcumin or celecoxib+curcumin. Collagen induced arthritis features is next evaluated by such anti-inflammatory parameters as characterization of the site of lesion lymphokine secretion profile (TNF-a, IL-1b, IL-6, IL-10, INFg) in the animal serum

1. A method of reducing a dosage size of an at least one NSAID in the treatment of a patient in need of an NSAID therapy, comprising simultaneous or step-wise administering of curcumin and said at least one NSAID, the curcumin being in an amount sufficient to reduce the NSAID concentration needed while maintaining the same therapeutic effect as compared to administering the NSAID alone.

2. The method according to claim 1, wherein said NSAID is selected from ketorolac, nabumetone, salsalate, diclofenac, indomethacin, nabumetone, phenylbutazone, oxyphenbutazone, dipyrone, ramifenazone, tenoxicam, valdecoxib, parecoxib, etoricoxib, celecoxib, sulindac, sulindac sulfide, exisulind, ibuprofen, naproxen, naproxen sodium, rofecoxib, nimesulide, aspirin, tolmetin, fenoprofen, flurbiprofen, loxoprofen, vedaprofen, meclofenamic acid, meclofenamate sodium, tolfenamic acid, acetaminophen, flunixin, piroxicam, oxaprozin, meloxicam, ketoprofen, etodolac, diflunisal and the like or any derivative, analogue, salt or prodrug thereof.

3. The method according to claim 2, wherein said drug is celecoxib, nimesulide, sulindac or sulindac sulfide or derivatives, analogues, salts or prodrugs thereof.

4. The method according to claim 2, wherein said drug is not celecoxib or derivatives, analogues, salts or prodrugs thereof.

5. The method according to claim 1, wherein said curcumin is a curcumin analogue or derivative selected from demethoxycurcumin and bisdemethoxycurcumin.

6. A method for treating an inflammatory disease or disorder in a patient, said method comprising administering to said patient curcumin and at least one NSAID, the amount of curcumin and said NSAID being effective so that in combination they have an anti-inflammatory effect.

7. A method for treating cancer in a patient in need thereof, said method comprising administering to said patient curcumin and at least one NSAID, the amount of curcumin and said NSAID being effective so that in combination they have an anti-cancer effect.

8. The method according to claim 6, wherein the amount of said NSAID is less than the amount of an NSAID administered alone in order to obtain a similar anti-inflammatory or anti-cancer effect.

9. The method according to claim 6, wherein said curcumin and said NSAID are administered not within the same formulation.

10. The method according to claim 6 wherein said NSAID is celecoxib, nimesulide, sulindac or sulindac sulfide or derivatives, analogues, salts or prodrugs thereof.

11. The method according to claim 6, wherein said NSAID is a drug other than celecoxib, or a drug other than sulindac, or a drug other than sulindac sulfide or derivatives, analogues, salts or prodrugs thereof.

12. A method for inhibiting cancer cell growth, comprising contacting cancer cells with an effective amount of a formulation comprising curcumin and at least one NSAID.

13. The method according to claim 12, wherein said cells are contacted with a single formulation comprising both curcumin and at least one NSAID.

14. The method according to claim 12 wherein said cells are contacted with a formulation containing curcumin, followed by contacting with a second formulation containing said at least one NSAID.

15. The method according to claim 12 wherein said cells are contacted with a formulation containing said at least one NSAID, followed by contacting with a second formulation containing curcumin.

16. A method for the treatment of cancer comprising administering to an individual in need of an effective amount of curcumin and an effective amount of at least one NSAID, the amounts being such so as to yield a synergistic combined effect of the two.

17. A method for the reduction of inflammation comprising administering to a patient in need of as an anti-inflammatory treatment an effective amount of curcumin and an effective amount of at least one NSAID drug, the amounts being such so as to yield a synergistic combined effect of the two.

18. A pharmaceutical composition comprising curcumin and at least one NSAID drug and a pharmaceutically acceptable carrier, excipient or diluent.

19. A pharmaceutical composition for the treatment of cancer, said composition comprising curcumin and at least one NSAID drug and a pharmaceutically acceptable carrier, excipient or diluent.

20. The composition according to claim 19, wherein said cancer is selected from leukemia, carcinomas, sarcomas, cancers of the brain, breast, cervix, colorectal, colon, head and neck, kidney, lung, non-small cell lung, melanoma, mesothelioma, ovary, sarcoma, stomach, uterus and Medulloblastoma.

21. The composition according to claim 20, wherein said cancer is colorectal or colon cancer.

22. A pharmaceutical composition for the treatment of inflammatory diseases or disorders, said composition comprising curcumin and at least one NSAID drug and a pharmaceutically acceptable carrier, excipient or diluent.

23. The pharmaceutical composition according to claim 22, wherein said inflammatory disease or disorder is selected from arthritis, rheumatoid arthritis (RA), spondyloathopathies, gouty arthritis, osteoarthritis, systemic lupus erythematosus and juvenile arthritis, acne vulgaris, acute respiratory distress syndrome, Addison's disease, allergic rhinitis, allergic intraocular inflammatory diseases, ANCA-associated small-vessel vasculitis, athrosclerosis, atopic dermatitis, autoimmune hemolytic anemia, autoimmune hepatitis, Behcet's disease, Bell's palsy, cerebral ischemia, Cogan's syndrome, dermatomyositis and psoriatic arthritis.

24. The pharmaceutical composition according to claim 23, wherein said inflammatory disease or disorder is arthritis.

25. The composition of claim 20 further comprising one or more additional active ingredients, selected from antibiotics, conventional anti-cancer, anti-inflammatory agents and other agents suitable for combination therapy.

26. The composition according to claim 20, wherein said curcumin is in an amount sufficient to reduce the amount of said at least one NSAID needed while maintaining the same therapeutic effect as compared to administering the NSAID alone.

27. The composition according to claim 26, for the treatment of any such disease or disorder treated by at least one NSAID.

28. A pharmaceutical composition for the treatment of cancer, comprising an effective amount of at least one NSAID, to be administered following treatment with curcumin.

29. A pharmaceutical composition for the treatment of inflammation, comprising an effective amount of at least one NSAID, to be administered following treatment with curcumin.

30. A pharmaceutical composition for the treatment of cancer, comprising an effective amount of curcumin, to be administered following treatment with at least one NSAID.

31. A pharmaceutical composition for the treatment of inflammation, comprising an effective amount of curcumin, to be administered following treatment with at least one NSAID.

32. A combination of two pharmaceutical compositions including a first composition comprising an effective amount of at least one NSAID drug, and a second composition comprising an effective amount of curcumin, the combination is intended for administering to a subject for treatment of cancer or inflammation, in which treatment said second composition is administered after administering said first composition.

33. A prophylactic formulation comprising curcumin and at least one NSAID and a pharmaceutically acceptable carrier, excipient or a diluent.

34. The prophylactic formulation according to claim 33 for use in the prevention of cancer or in the reduction of the likelihood of contracting cancer in a subject susceptible to contracting said disease.

35. Use of curcumin and at least one NSAID for the preparation of a pharmaceutical composition.

36. The use according to claim 35, wherein said composition is used for the treatment of cancer or inflammation.

37. The use according to claim 35, wherein said curcumin is in an amount sufficient to reduce the amount of said at least one NSAID needed while maintaining the same therapeutic effect as compared to administering said NSAID alone.

38. Use of at least one NSAID for the preparation of a pharmaceutical composition for the treatment of cancer or inflammation in a subject, said composition is to be administered following treatment with curcumin.

39. Use of at least one NSAID for the preparation of a pharmaceutical composition for the treatment of cancer or inflammation in a subject, said composition is to be administered simultaneously with treatment with curcumin.

40. Use of curcumin for the preparation of a pharmaceutical composition for the treatment of cancer or inflammation in a subject, said composition is to be administered following treatment with at least one NSAID.

41. Use of curcumin for the preparation of a pharmaceutical composition for the treatment of cancer or inflammation in a subject, said composition is to be administered simultaneously with treatment with at least one NSAID.

42. A kit or a commercial package comprising a dosage unit of the composition of claim 18.

43. A kit or a commercial package comprising a dosage unit of the vaccine of claim 33.

44. The kit of claims 42, wherein said composition is in a single dosage unit.

45. The kit of claims 42, wherein said composition is in a number of different dosage forms.
